(19) United States
(12) Patent Application Publication
(10) Pub. No.: US 2007/0218114 A1
(43) Pub. Date: Sep. 20, 2007

(54) Soluble Strip for Oral or Topical Administration

(75) Inventors: Alex Duggan, Epsom (GB); Jean-Loic Baratoux, Courbevoie (FR)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Passionfor Life Healthcare Limited, Epsom (GB)

(21) Appl. No.: 11/570,493

(22) PCT Filed: Jun. 13, 2005
(86) PCT No.: PCT/GB05/02330

§ 371 Date: Feb. 21, 2007

(30) Foreign Application Priority Data
Jun. 12, 2004 (GB) 0413138.9
Mar. 23, 2005 (GB) 0506018.1

(51) Int. Cl. A61K 9/00; A61K 45/00; A61P 1/02; A61P 17/02; A61K 45/06

(52) U.S. Cl. 424/443; 424/725

(57) ABSTRACT

A composition for oral administration comprising at least one active ingredient including at least one active ingredient having activity in reducing the effect of conditions of the throat or throat disorders, distributed within a soluble base material, the composition being provided as a strip for inter-oral administration, method for the preparation thereof and use thereof in alleviating throat conditions or throat disorders.

The invention relates to a soluble composition for the administration of an active ingredient to a site on the mucus membranes of the throat of a human or animal subject, having at least an activity which minimises the effects of snoring and/or sleep apnoea and/or which is effective in blocking adhesion of harmful bacteria to host tissues and/or in oral care or for treating conditions of the throat or throat disorders; and a composition for topical administration of an active ingredient to an open skin condition in a human or animal subject, having at least an activity in open skin or wound repair or healing and/or which is effective in blocking adhesion of harmful bacteria to host tissues. The invention also relates to a method of preparation of such a composition.

Throat sprays are known as a means to deliver a composition intended to reduce the impact of snoring in a subject, particularly to attenuate the noise of the snore, and to reduce its impact, both in disturbing sleep and as perceived by other parties, rather than to treat any underlying condition as such. Such compositions comprise one or more lubricant active ingredients intended to keep the soft tissues and mucous membranes of the nose and pharynx moist and lubricated and thus reduce the noise associated with snoring which arises in particular from the soft tissues of the throat.

Various compositions exist for this purpose. These typically include a mixture of active nature oils. Compositions may include additional function other than that of keeping the mucous membrane moist, for example including active ingredients having decongestant properties. Some compositional ingredients do not however lend themselves well to an aerosolisable form. Sprays moreover need mechanical delivery systems, which can fail, and if not operated correctly can fail to deliver an accurately measured dose. Some spray systems, particularly nasal sprays, can induce irritation which might lead to sneezing or coughing and loss of active ingredient. Alternative delivery systems for example oral suspensions are available for direct oral administration, for example as a gargle or mouthwash, however the limited period of contact between liquid and throat can limit effectiveness of transfer of active ingredient.

A second problem generic to throat and mouth sprays and compositions for treating open skin and wounds is that the active compositions can be limited in effectiveness, particularly over time. It is inherent in the nature of the problem that they are intended to solve that sustained activity over a sustained period, and most preferably overnight in the case of snoring, is desirable. However in practice the effect of spraying or otherwise applying lubricants on to the mucous membranes of the nose or throat is likely to be more short lived as the active ingredient is rapidly lost from the desired site through for example evaporation, the action of secreted nasal mucus and saliva etc. Similarly the effect of active compositions for open skin or wounds is likely to be short lived due to accidental contact with surroundings or absorption by dressings, weeping and the like.

We have now surprisingly found that active compositions can be applied to the mouth, throat, open skin or wounds, in manner to stabilise the active ingredients in situ on the mucous membranes of the throat or on the wound for a sustained period of time, and in manner to overcome additional problems associated with known delivery systems.

Accordingly in the broadest aspect of the invention there is provided a soluble composition comprising at least one active ingredient distributed within a soluble base material, wherein the active ingredient has:
at least an activity which minimises the effects of snoring and/or sleep apnoea and/or in blocking adhesion of harmful bacteria to host tissues to minimise the effects of conditions of the throat or throat disorders and/or for oral care and the composition is provided as a strip for inter-oral administration of the active ingredient to a site on the mucus membranes of the throat of a human or animal subject; or
at least an activity in open skin or wound repair or healing and/or in blocking adhesion of harmful bacteria to host tissues and the composition is provided as a strip for topical administration of the active ingredient to an open skin condition or wound in a human or animal subject.

In accordance with one embodiment of the invention at least one active ingredient is provided which has an effect in reducing the effects of conditions of the throat or throat disorders such as bacterial infection, strep throat, sore throat. Strep throat is an infection of the pharynx caused by Streptococcus pyogenes. The symptoms of strep throat, white patches in the tonsils, swollen lymph nodes in the neck, fever and increased temperature, headache, loss of appetite, fatigue, may occur. It is typically treated with antibiotics. Active ingredients are suitably active when in situ on the mucosa of the throat.

Preferably such at least one active ingredient is selected from an ingredient which has an effect in blocking adhesion of harmful bacteria to host tissues, more preferably selected from extracts of plant or animal such as aloe, in particular aloe vera, such extracts comprising polysaccharides derivable from the plant or animal and mixtures thereof, preferably such extract comprises a combination of polysaccharides comprising 60-100% of D-mannose, 40-0% of D-glucose and 0-10% of other monosaccharides, more preferably known from PCT/NL02/00868, and may be negatively charged, alternatively a suitable bacteria blocking extract may be selected such as xylitol or equivalents, oligosaccharides which serve as decoys and occupy bacteria's carbohydrate binding such as pentasuccharide (milk), and high molecules polysaccharide such as cranberry (see site of Cranberry Institute). The composition may additionally include any known active ingredient for treating conditions of the throat or throat disorders.

In accordance with a further embodiment of the invention at least one active ingredient is provided which has an effect on oral care conditions such as bad breath, halitosis, gingivitis and the like. Preferably such at least one active ingredient is selected from an ingredient which has an effect in blocking adhesion of harmful bacteria to host tissues, as hereinbefore defined.

In accordance with a further embodiment of the invention at least one active ingredient is provided which has an effect in reducing the effects of snoring and/or in reducing sleep apnoea when in situ on the mucosa of the throat. Preferably such at least one active ingredient is selected from moisturizer or humectant such as for example hyaluronic acid, glycerol, sorbitol, PEG;

decongestant such as for example Hyaluronic acid, Calendula officinalis flower extract, Thymus vulgaris, Menthyl lactate, Mentha piperita (or any other mint/peppermint derivative or extract), Lavendula augustifolia (or any other lavender derivative or extract), Phenylephrine hydrochloride, Pseudephedrine, Ascorbic acid (vitamin C), Acerola, Rumex crispus (yellow dock), Eucalyptus globulus (eucalyptus oil), Levmetamfetamine, Oxymetazoline hydrochloride, Propylhexedrine, Xylometazoline hydrochloride, menthol, eugenol, cineol, rosemary oil (rosmarinus), summer savory oil (satureia hortensis), wild thyme oil (thymus serpyllum), firtree oil, lavendula vera oil, geranium oil, cinnamon oil, Hawthorn extract (crataegus oxyacantha), rose hips extract (rosa canina), cypress oil (cupressus sempervirens), salts such as for example Zinc Gluconate and combinations thereof;

gums for example selected from Xanthan gum, Cellulose gum, Guar hydroxy-propyltrimonium chloride, gellan gum, alpha-glucan, carrageenan, pectin, sclerotium, alginate, chitosan;

pharmaceutical and cosmetic grade polymers such as for example PVP (polyvinylpyrrolidone);

derivatives of polysaccharides as hereinbefore defined; and

a mixture of lubricating and/or moisturising oils for example selected from the group comprising Calendula officinalis flower extract, Olivum (olive oil), Helianthus annus (sunflower oil), Prunus dulcis (sweet almond oil), Sesamum indicum (sesame oil), Aloe vera, Aloe barbadensis, Euphorbium officinarum, Lactoperoxidase and combinations thereof, Thymus vulgaris, Menthyl lactate, Mentha piperita (or any other mint/peppermint derivative or extract), Lavendula augustifolia (or any other lavender derivative or extract), Phenylephrine hydrochloride, Pseudephedrine, Acerola, Rumex crispus (yellow dock), Eucalyptus globulus (eucalyptus oil), menthol, eugenol, cineol, rosemary oil (rosmarinus), summer savory oil (satureia hortensis), wild thyme oil (thymus serpyllum), firtree oil, lavendula vera oil, geranium oil, cinnamon oil, oregano oil, vervain oil, clove oil, cedar oil, licorice, Hawthorn extract (crataegus oxyacantha), rose hips extract (rosa canina), cypress oil (cupressus sempervirens) and combinations thereof;

and wherein the composition may additionally include an active ingredient which has an effect in blocking adhesion of harmful bacteria to host tissues, as hereinbefore defined.

In accordance with a further embodiment of the invention at least one active ingredient is provided which has at least an activity in open skin or wound repair or healing, for example anticoagulants, or treatments for burns, acne, boils and the like, which has an effect in blocking adhesion of harmful bacteria to host tissues, as hereinbefore defined. The composition may additionally include an active ingredient selected from known anticoagulants, or treatments for burns, acne, boils and the like, preferably from calcium alginate, gelatin, regenerated oxidised cellulose (calcium or sodium salt of copolymer of anhydroglucose and anhydroglucuronic acid), “microfibrillaire” collagen, adrenalin, thrombin, iron chloride, sodium alginate/hyaluronic acid, vasopressine, desmopressine acetate, aprotinine, epsilon-aminocaproic acid, tranexamic acid and the like.

The base material comprises a carrier which is conformed as a strip to serve as a delivery system for a measured dose of active ingredient and which is impregnated with, coated with or otherwise carrying the active ingredient(s) to enable the distribution of this active ingredient. For example a base material of a rapidly dissolving inter-oral film of the type is known for example in relation to breath freshening strips. As the base material dissolves, under action of moisture in the mouth or on open skin or wound, a controlled quantity of the active ingredient is released and is delivered to the soft tissue at the back of the throat or to the open skin or wound where it is able to have the desired effects.

Reference herein to a strip is to any soluble prolonged release presentation of the composition which is conformable and is adapted to lie in a subjects mouth without causing obstruction or interfering with breathing, talking or swallowing or the like, or to conform to the surface of a subjects open skin or wound. Preferably the strip comprises a flexible film or the like.

In use, the strip to be placed in a subject's mouth is intended to be placed at the back of the throat, near to the desired area of operation. The strip is particularly suited to delivery of ingredients having activity in relation to snoring or apnoea, by delivery to the mucosa of the throat, in particular at the soft tissue in the pharyngeal region of the back of the throat, to keep the pharyngeal membranes moist and lubricated.

The strip is conformed as a relatively thin planar structure to facilitate rapid inter-oral dissolution. For example, the strip is no more than 20 to 250 micron thick, more preferably in the range 40-100 micron thick. The strip may be of any suitable shape, for example being square or rectangular for ease of storage, and is preferably generally planar and approximately 0.5 to 2 cm in length and breadth.

The strip is manufactured from a material which is rapidly soluble within the subject's mouth under the action of saliva and oral enzymes, or on open skin or wounds under the action of tissue fluids. The base material for the strip is any suitable soluble solid material, which term includes gel-like and other materials which are sufficiently solid to enable the strip to be conformed to its desired shape.

In particular, the carrier or base material of the strip comprises a soluble gel material, and is for example based upon on an organic gel, which could for example be a fish, animal, bovine or marine gelatine or vegetal gelatine-like product, a polysaccharide, a cellulosic material, pectin such as from fruits, or other suitable base. Other materials may be added to the base gel, for example to stabilise, add other effects flavour etc. The carrier or soluble base material may be inert, or may itself have an activity or other desired property, whether in relation to the primary purpose of the invention or otherwise.

The composition may include additional active ingredients, including a plurality of active ingredients having an activity in relation to the condition or the throat or throat disorder, oral conditions, or conditions of snoring and/or sleep apnoea, open skin or wound healing or repair agents and/or active ingredients having other desired activity.

These active ingredients include at least one active ingredient with physical (moisturising, lubricating, cooling etc) or pharmacological (for example decongestant, anti-histamine, anti-bacterial, anti-inflammatory, analgesic etc) activity. For example active ingredients might include ingredients having any desired physical or pharmacological activity on the mucous membranes of the throat, including without limitation decongestants, lubricants, antibacterial and antiseptic compositions, anti-histamines, anti-inflammatory compositions, analgesics, and other medicaments and non-medicaments. Additional ingredients may include breath-fresheners and deodorisers. Inactive ingredients might further be added in suspension or solution for example to stabilise or preserve the soluble base, balance the pH of the base, bring the base to closer approximation to isotonic concentration etc. The composition may additionally include adjuvants and the like such as vitamins for example selected from Ascorbic acid (vitamin C) which enhance the active ingredient effect. The composition may include additional ingredients for formulation purpose, for example selected from sodium chloride which maintains favorable isotonicity.

In the preferred embodiment the active ingredient comprises one or more lubricant/moisturisers to lubricate and/or moisturise the throat membranes. Natural oils and the like are especially preferred ingredients. For example, the active ingredient may comprise a mixture of lubricating and/or moisturising oils selected from the group comprising: Hyaluronic acid, Glycerol, Calendula officinalis flower extract, Guar hydroxypropyltrimonium chloride, Xanthan gum, Cellulose gum, Olivum (olive oil), Helianthus annus (sunflower oil), Prunus dulcis (sweet almond oil), Sesamum indicum (sesame oil), Aloe vera, Aloe barbadensis, Euphorbium officinarum, Oxymetazoline hydrochloride, Lactoperoxidase and combinations thereof.

Additionally or alternatively the composition preferably comprises as an active ingredient at least one decongestant, being an ingredient having a chemical or pharmacological or other effect of reducing airway congestion and/or limiting further airway mucus production. In particular, the additional ingredient comprises a nasal decongestant selected to clear and/or limit the further production of nasal mucus. The active ingredient may be a natural oil, a pharmacologically active synthetic preparation, or combination. Suitable examples include: Hyaluronic acid, Calendula officinalis flower extract, Thymus vulgaris, Menthyl lactate, Mentha piperita (or any other mint/peppermint derivative or extract), Lavendula augustifolia (or any other lavender derivative or extract), Phenylephrine hydrochloride, Pseudephedrine, Ascorbic acid (vitamin C), Acerola, Rumex crispus (yellow dock), Eucalyptus globulus (eucalyptus oil), Levmetamfetamine, Oxymetazoline hydrochloride, Propylhexedrine, Xylometazoline hydrochloride, Zincum Gluconicum, menthol, eugenol, cineol, rosemary oil (rosmarinus), summer savory oil (satureia hortensis), wild thyme oil (thymus serpyllum), firtree oil, lavendula vera oil, geranium oil, cinnamon oil, Hawthorn extract (crataegus oxyacantha), rose hips extract (rosa canina), cypress oil (cupressus sempervirens) and combinations thereof.

Alternatively the active ingredient may comprise an antibiotic composition that has bacteria blocking compositions. By reference to PCT/NL02/00868, the contents of which are incorporated by reference, we emphasise the role of bacteria blocking active ingredients.

Preferably an active ingredient effective in blocking harmful bacteria is effective in blocking adhesion of such bacteria to host tissues. Active ingredients are known which are effective in preventing adhesion of harmful bacteria to host tissues, and in particular extracts of plant or animal such as aloe, in particular aloe vera, such extracts comprising polysaccharides derivable from the plant or animal and mixtures thereof. Preferably such extract comprises a combination of polysaccharides comprising 60-100% of D-mannose, 40-0% of D-glucose and 0-10% of other monosaccharides. Suitably such a bacteria blocking extract is known from PCT/NL02/00868, and is negatively charged. Alternatively a suitable bacteria blocking extract may be selected such as xylitol or equivalents, oligosaccharides which serve as decoys and occupy bacteria's carbohydrate binding such as pentasuccharide (milk), and high molecules polysaccharide (cranberry, see site of Cranberry Institute).

It is a particular advantage that the antibiotic composition of the invention may prevent inflammation and bacterial infections by bacteria such as MRSA (Methicillin Resistant Staphylococcus Aureus) and the like.

Examples of infections of the throat or of open skin or wounds which can be prevented and treated by anti adhesive polysaccharides of the present invention include those caused by:
Staphylococcus epidermis which plays an important role in the pathogenesis of some forms of endophthamitis occurring after cataract surgery
Moraxella bovis as the source of infectious bovine keratoconjunctivitis
Staphylococcus aureus which adheres to the skin and mucous tissues
bacteria involved in Otitis media and nasopharyngal infections such as Haemophilus influenza, Streptococcus penumoniae and Moraxella catarrhalis the oral cavity, wherein the dental plaque biofilm plays a pivotal role in the progression of dental diseases and polysaccharides are of great importance in the ecology of the dental biofilm, caused by bacteria involved in caries such as Streptococcus sobrinus as acariogenic strain, Streptococcus mutans, Streptococcus salivarius, Streptococcus gordonii and Actinomyces viscosus, Actinobacillus actinomycetecomitans
periodontopathogenic bacteria such as Porphyromonas gingivalis and Streptococcus salivarius, Streptococcus oralis, Fusobacterium nucleatum and Prevotella intermedia
All oral spirochetes which are classified in the genus Treponema, such as denticola, pectinovorum, socranskii and vincentii,
Mycoplasma salivarium
microorganisms involved in nasal polyposis
microorganisms involved in Sinusitis

Suitably the composition present in the oral strip of the invention comprises active ingredient present in any effective amount, preferably in the range 1-20 wt % and for example 2-10 wt %. For example the polysaccharide constituent may be a negatively charged mixture of 70-90% D-Mannose 30-10% glucose, and 0-10% of other monosaccharides and is a plant derivative, for example Aloe Vera derivative, more preferably 2QR as is known from PCT/NL02/00868.

In an embodiment, the composition incorporates the nasal decongestant as above described together with at least one lubricant and/or moisturiser. This is particularly effective. Systems which rely on lubrication and/or moisturising alone will be entirely ineffective against nasal snoring where a subject has an infectious, irritated or allergic breathing congestion, and such infectious, irritated or allergic breathing congestion is likely to exaggerate the undesirable effects of nasal snoring. For the same reason, a composition may also include an active ingredient with anti-histaminic action.

In a preferred embodiment the composition further includes means to stabilise the active ingredients in situ on the mucosa of the subject's throat or open skin or wounds and to retain activity levels over a sustained period of time. For example, it is desirable to provide for measurable activity, e.g. at least 50% of initial base line activity level, for a sustained period of 4 or more hours, and ideally of 6 to 12 hours, to give overnight effectiveness.

This can be achieved in that the active ingredients are selected to be chemically structured and/or associated with other molecular or physical structures that facilitate adhesion of the active ingredients on the mucosa of the throat. For example, the active ingredients are molecularly structured as, or associated with, polar structures, and in particular polar structures presenting a positive surface charge, which are effective in adhering to the generally negatively charged membranes of the throat.

The use of additional ingredients within the composition to provide for chemical binding, and for example for the use of liposome technology, will be familiar. However, in accordance with a particular preferred embodiment of the invention a part or all of the active ingredients are encapsulated within encapsulation structures selected to provide the said degree of adhesion to the mucous membranes of the throat, and adapted to release the active ingredients slowly over time in situ. These encapsulation structures are distributed within the base material in the strip composition.

In a particularly preferred embodiment, the encapsulation structures comprise multilamellar microparticles. The multilamellar microparticles are selected to exhibit good adhesion to the mucous membranes of the throat, and are small enough to be effectively distributed in the strip. The multiple layers are structured to give slow release of the active ingredient over the desired time period, so that a single strip dose gives sustained activity over time, for example providing for measurable activity for a sustained period of four or more hours, and ideally of for example 6 to 12 hours, to give overnight effectiveness.

The microparticles are sized and shaped to form an effective distribution within the base material in the strip as a composition in accordance with the invention. The microparticles in particular comprise generally spherical particles or microspheres. Particle sizes in the range 0.1 to 50 μm, and for example 1 to 20 μm are likely to be preferred. Particle levels of 5-25% within the composition are likely to be suitable.

The microparticles are adapted to facilitate slow release of the active ingredients over time, and are found inherently to show good adhesion to the mucous membranes of the throat, and may adhere also to tissue of open skin or wounds. The net result of this is that the active ingredients are stabilised in situ on the mucous membranes at the back of the throat, and that the active ingredients are then released steadily at the site were they are required. Loss of activity over time is significantly reduced compared with conventional compositions relying for example on liposome technology, and it becomes possible to maintain reasonable levels of activity over the sort of time scale necessary to be effective overnight, and for example to assist in providing a relatively less disturbed night's sleep.

In particular the microparticles comprise multiple layered structures formulated with one or more of and preferably examples of all of:

surfactant layers (comprising any type of surfactant such as anionic, non-anionic, cationic, phospholipids and the like such as sucroesters and guar hydroxypropyltrimonium chloride), and hydrophobic or lipophilic materials such as aliphatic and aromatic hydrocarbons, optionally halogenated, higher alcohols, ketones and the like, for example including Vitamins (A, E, D), carotenoides, polyphenols, vegetable oils, essential oils, phytosterols, lipophilic preservatives, menthol, linalool, eucalyptol, and the like; and polar layers including solvents or polar media such as water, glycerol, PEG, sorbitol, glycol, hydrophilic materials such as alcohols or ethoxylated alcohols, carboxylic acids or salt of a fatty acid, quaternary ammonium derivatives, sulphonates or sulphates and the like, vitamins (B, C), flavonoides, 18-beta glycyrrhetinic acid and derivatives, glycerol, active ingredients such as plant extract as hereinbefore defined; hydrophilic preservative; cellulose polymer, hyaluronic acid and derivatives, alpha-hydroxide acid, and the like.

The positively charged polymer is preferably selected from in oral strips:

Pectin; cellulose; sodium hyaluronate; guar hydroxypropyltrimonium chloride; polysorbate 60, and optionally additionally cellulose; xanthan gum; chitosan or quaternary ammonium.

Preferably the composition comprises:

Solvent 30-60%,

Humectant 8-14%,

Texturant 0-2%,

Preservative 0-2%,

Acidity regulator 0-1%,

And microparticles as hereinbefore defined 10-50%

The microparticles thus preferably comprise multi-lamellar structures of surfactant layers, which are able to encapsulate active ingredients to a very high degree for protection and controlled slow release. The surfaces of the microparticles are such as to be adapted to enhance adhesion to human skin, and hence to fix the particles in position on the mucous membranes of the throat as the active ingredients are progressively released.

Suitable compositions include 30 to 50% surfactant, 30 to 50% polar medium, and 10 to 60% active binding agent, comprising hydrophilic and hydrophobic agents as appropriate.

Microparticles providing the controlled binding for slow release of physically active ingredients at the active site on the mucous membrane of the throat of the human, non-human mammal or other animal subject to provide an active effect are found to be surprisingly effective for such an application, both because the microparticles bind effectively to the membranes to ensure good delivery in situ, and because they lend themselves ideally to the controlled slow release of the microencapsulated active ingredients.

Adsorbed inside the layers of the microparticles are one or more different active ingredients as hereinbefore defined.

The microparticles fix the active ingredients adsorbed within each shaped layer in position on the mucous membranes of the user, protect the active ingredients and slowly release them in situ, and might also assist in providing a desired lubricating effect.

The composition in this preferred embodiment thus comprises a dispersion of multilayer microparticles as above described having active ingredients encapsulated within the layers thereof and dispersed within a soluble base so as to be conformable as a strip for application.

Further active or inactive ingredients might be provided either encapsulated within the microparticles or separately in suspension or solution within the base for various purposes.

In accordance with a further aspect of the invention there is provided a method of preparing a composition for the controlled delivery of an active ingredient over time in situ at the mucous membranes of a human, non-human mammal or other animal comprises the steps of distributing at least one active ingredient, including at least one active ingredient having an activity to reduce the effects of a throat condition or throat disorder such as for example snoring and/or sleep apnoea, within a soluble base, and providing as a strip of material for inter-oral administration.

Preferably the method comprises the steps of introducing a dose of an active ingredient and any other components as hereinbefore defined into a base carrier material. The method may comprise combining the active ingredient and other components in solution with a solution of the base material and casting as a film to dry to a strip. Drying may be by evaporation or the like. Alternatively a strip may be impregnated or coated with a dose of active ingredient and other components. Strips may be coated with other components as desired. Suitably strips are made up in bulk films which are subsequently cut to size and shape as hereinbefore defined.

In the particular preferred embodiment, the method includes the step of:

microencapsulating at least one ingredient having the said activity on the mucosa of the subject within the layers of a multilamellar microparticle; distributing a plurality of such particles in the soluble base.

In accordance with the further aspect of the invention, a method of delivering an active ingredient to the throat of a human, non-human mammal or other animal subject for controlled release over time in situ at the mucous membranes of the subject comprises the fabrication of a composition as above described and its administration to the subject.

The active ingredient may have a therapeutic effect, for example exhibiting pharmacological or other physiological activity, or may have a non-therapeutic effect, for example in the reduction of the social effects of snoring and the like. Thus in one alternative, the method applies an active ingredient which is directly physiologically or pharmacologically active to treat a specified medical condition and therefore comprises a method of medical treatment. In another alternative the method comprises the application of an active ingredient which has a physical non-medical activity not specifically being a method of medical or therapeutic treatment.

In a particular example of the latter, an active ingredient comprises a lubricant and/or moisturiser to lubricate or moisturise the mucous membranes of the throat of a user, in particular of the soft tissue in the pharyngeal region to minimise the effects of snoring. The method thus serves not to treat any underlying condition which might be contributing to the snoring as such, but rather to attenuate the noise produced thereby and so provide relief to third parties from the anti-social effects of the noise associated therewith.

In a further aspect of the invention there is provided a strip as hereinbefore defined for use in the alleviation of throat conditions or throat disorders as hereinbefore defined.

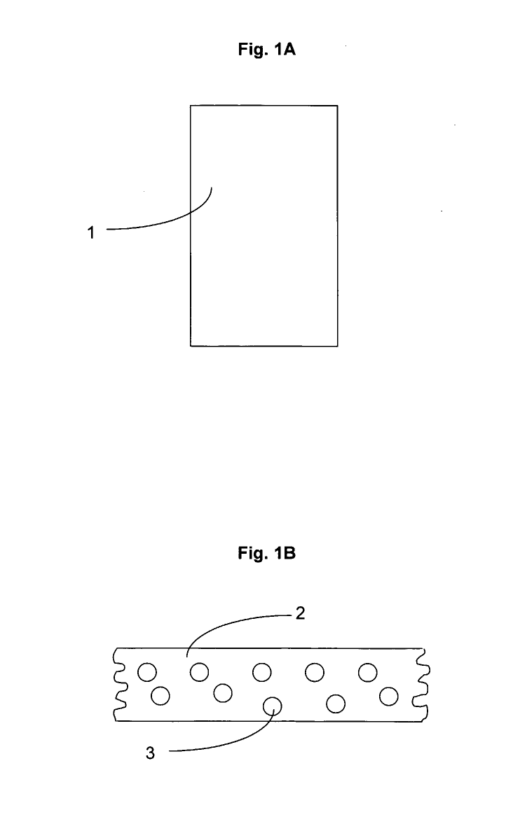

By way of example only, FIG. 1 illustrates a strip composition in accordance with the invention.

Further by way of example, an example composition is described.

A schematic representation of a possible strip in accordance with the invention is shown in FIGS. 1a and 1b.

FIG. 1a illustrates the strip in plan view (1). A more detailed crossed sectional view is illustrated in FIG. 1b. The composition of the strip is visible herein, as consisting of a gelatine basis (2), which in the example makes use of fish gelatine, but could equally well use porcine, bovine or alternative animal gelatine, marine gelatine, vegetal gelatine-like substance, or a cellulose or other type of vegetarian base. The base material (2) forms the basis of the strip (1) which serves as a rapid-dissolve inter-oral film of the type which is known in relation to breath freshening applications.

The active ingredient is encapsulated within the layers multilamellar microspheres (3). In use, the strip is placed near to the back of the throat, in the vicinity of the active site. The gelatine (2) dissolves rapidly under the action of the subject's saliva once the strip (1) has been inserted orally. This frees the microspheres (3) to locate on the desired site on the mucosa in the throat, especially at the soft tissues in the pharyngeal region. This effectively locates the ingredient in situ. The strip delivery system is singularly effective in maximising delivery of the active component (the microspheres) to the active site. Contact over a sustained period as the strip dissolves ensures that the microspheres have the opportunity to adhere to the target membranes to a greater extent than might be the case for other delivery systems. The spheres, once so fixed, effect the desired slow release of active ingredient, for the treatment of the effects of snoring and/or sleep apnoea.

Mechanistically, this is effected conveniently in that the microsphere has a multilamellar structure and traps active ingredients between each layer. The microsphere body is positively charged, for example by making use of a food cationic polymer, and therefore adheres to the mucosa in the throat as the tissue in the throat is negatively charged. Slowly, over time, the layers of the microsphere are dispersed by the mucosal enzymes and fluid, releasing the active ingredients as they do so, and providing for an effective time-release system.

### EXAMPLE 1

| | |
|---|---|
| GELATINE | % |
| MINT OIL | % |
| EUCALYPTUS OIL | % |
| OLIVE OIL | % |
| WATER | % |
| SPHERULITE PARTICLES | % |

SPHERULITES contain:

| | |
|---|---|
| Water | 62.18% |
| Sucroesters and Guar Hydroxypropyltrimonium | 25.00% |
| Olive Oil | 2.00% |
| Mint Oil | 3.00% |
| Grapeseed Oil | 1.50% |
| Tocopherol Acetate | 1.00% |
| Glycerin | 5.00% |
| Potassium Sorbate | 0.15% |
| Citric Acid | 0.07% |
| Sodium Hyaluronate | 0.10% |

### EXAMPLE 2

| | |
|---|---|
| Pectin | 35-42% |
| glycerol | 16-22% |
| surfactants (sorbitan stearate, polysorbate 60) | 0-14% |
| aqua | 0-10% |
| flavours (peppermint flavour, peppermint oil, menthol | 6-8% |
| powder) | |
| rapeseed oil | 5-7% |
| cellulose | 5-7% |
| olive oil | 0-2% |
| grapeseed oil | 0-1.5% |
| dl-alpha tocopherol acetate (vitamin E) | 0.3-2% |
| sweeteners (*aspartame, sodium saccharin, acesulfame-K) | 0.4-0.9% |
| preservatives (potassium sorbate) | 0.3-0.9% |
| citric acid | 0-0.2% |
| sodium hyaluronate | 0-0.1% |
| guar hydroxypropyl trimonium chloride | 0-0.02% |
| colour (FCF blue) | 0-0.01% |
| some of the above may be present | 0-22% |
| in the form of optional spherulites | |

*a source of phenylalanine

### EXAMPLE 3

Purified water

| | |
|---|---|
| Glycerin | 8-15% |
| Solubilised anise oil component | 0-0.5% |
| Ampiphilic humectant (pentylene glycol) | 0-8% |
| Xanthan gum | 0.05-0.18% |
| Acidifier (citric acid) | 0.08-0.15% |
| Avicel PC611 | 0.8-1.4% |
| Preservatives (sodium benzoate, potassium sorbate) | 0.3-0.6% |
| 2QR | 4-7% |
| 2QR in spherulites | 5-14% |

The compositions are examples only illustrative of but not limiting on the overall scope of the invention.

1. A soluble composition comprising at least one active ingredient distributed within a soluble base material, wherein the active ingredient has:
at least an activity which minimises the effects of snoring and/or sleep apnoea and/or in blocking adhesion of harmful bacteria to host tissues to minimise the effects of conditions of the throat or throat disorders and/or for oral care and the composition is provided as a strip for inter-oral administration of the active ingredient to a site on the mucus membranes of the throat of a human or animal subject; or
at least an activity in open skin or wound repair or healing and/or in blocking adhesion of harmful bacteria to host tissues and the composition is provided as a strip for topical administration of the active ingredient to an open skin condition or wound in a human or animal subject.

2. The composition of claim 1 comprising at least one active ingredient which has an effect in blocking adhesion of harmful bacteria to host tissues thereby reducing the effects of conditions of the throat or throat disorders such as bacterial infection, strep throat, sore throat when in situ on the mucosa of the throat.

3. The composition of claim 1 wherein such at least one active ingredient is selected from an ingredient which has an effect in blocking adhesion of harmful bacteria to host tissues, more preferably selected from extracts of plant or animal such as aloe, in particular aloe vera, such extracts comprising polysaccharides derivable from the plant or animal and mixtures thereof, preferably such extract comprises a combination of polysaccharides comprising 60-100% of D-mannose, 40-0% of D-glucose and 0-10% of other mono saccharides, and may be negatively charged, alternatively a suitable bacteria blocking extract may be selected such as xylitol or equivalents, oligosaccharides which serve as decoys and occupy bacteria's carbohydrate binding such as pentasuccharide (milk), and high molecules polysaccharide such as cranberry; and wherein the composition may additionally include any known active ingredient for treating conditions of the throat or throat disorders.

4. The composition of claim 1 comprising at least one active ingredient which has an effect on oral care conditions such as bad breath, halitosis, gingivitis and the like wherein such at least one active ingredient is selected from an ingredient which has an effect in blocking adhesion of harmful bacteria to host tissues.

5. The composition of claim 1 comprising at least one active ingredient which has an effect in reducing the effects of snoring and/or in reducing sleep apnoea when in situ on the mucosa of the throat, wherein such at least one active ingredient is selected from:
moisturizer or humectant such as for example hyaluronic acid, glycerol, sorbitol, PEG;
decongestant such as for example Hyaluronic acid, Calendula officinalis flower extract, Thymus vulgaris, Menthyl lactate, Mentha piperita (or any other mint/peppermint derivative or extract), Lavendula augustifolia (or any other lavender derivative or extract), Phenylephrine hydrochloride, Pseudephedrine, Ascorbic acid (vitamin C), Acerola, Rumex crispus (yellow dock), Eucalyptus globulus (eucalyptus oil), Levmetamfetamine, Oxymetazoline hydrochloride, Propylhexedrine, Xylometazoline hydrochloride, menthol, eugenol, cineol, rosemary oil (rosmarinus), summer savory oil (satureia hortensis), wild thyme oil (thymus serpyllum), firtree oil, lavendula vera oil, geranium oil, cinnamon oil, Hawthorn extract (crataegus oxyacantha), rose hips extract (rosa canina), cypress oil (cupressus sempervirens), salts such as for example Zinc Gluconate and combinations thereof;
gums for example selected from Xanthan gum, Cellulose gum, Guar hydroxypropyl-trimonium chloride, gellan gum, alpha-glucan, carrageenan, pectin, sclerotium, alginate, chitosan;
pharmaceutical and cosmetic grade polymers such as for example PVP (polyvinylpyrrolidone);
derivatives of polysaccharides; and
a mixture of lubricating and/or moisturising oils for example selected from the group comprising Calendula officinalis flower extract, Olivum (olive oil), Helianthus annus (sunflower oil), Prunus dulcis (sweet almond oil), Sesamum indicum (sesame oil), Aloe vera, Aloe barbadensis, Eupborbium officinarum, Lactoperoxidase and combinations thereof, Thymus vulgaris, Menthyl lactate, Mentha piperita (or any other mint/peppermint derivative or extract), Lavendula augustifolia (or any other lavender derivative or extract), Phenylephrine hydrochloride, Pseudephedrine, Acerola, Rumex crispus (yellow dock), Eucalyptus globulus (eucalyptus oil), menthol, eugenol, cineol, rosemary oil (rosmarinus), summer savory oil (satureia hortensis), wild thyme oil (thymus serpyllum), firtree oil, lavendula vera oil, geranium oil, cinnamon oil, oregano oil, vervain oil, clove oil, cedar oil, licorice, Hawthorn extract (crataegus oxyacantha), rose hips extract (rosa canina), cypress oil (cupressus sempervirens) and combinations thereof;
and wherein the composition may additionally include an active ingredient which has an effect in blocking adhesion of harmful bacteria to host tissues.

6. The composition of claim 1 comprising at least one active ingredient which has at least an activity in open skin or wound repair or healing, for example anticoagulants, or treatments for burns, acne, boils and the like, which has an effect in blocking adhesion of harmful bacteria to host tissues.

7. The composition of claim 6 which additionally comprises an active ingredient selected from known anticoagulants, or treatments for burns, acne, boils and the like, preferably from calcium alginate, gelatin, regenerated oxidised cellulose (calcium or sodium salt of copolymer of anhydroglucose and anhydroglucuronic acid), microfibrillaire collagen, adrenalin, thrombin, iron chloride, sodium alginate/hyaluronic acid, vasopressine, desmopressine acetate, aprotinine, epsilon-aminocaproic acid, tranexamic acid.

8. The composition of claim 1
wherein base material comprises a carrier which is conformed as a strip to serve as a delivery system for a measured dose of active ingredient and which is impregnated with, coated with or otherwise carrying the active ingredient(s) to enable the distribution of this active ingredient

9. The composition of claim 1 wherein a strip is any soluble prolonged release presentation of the composition which is conformable and is adapted to lie in a subjects mouth without causing obstruction or interfering with breathing, talking or swallowing, or to conform to the surface of a subjects skin or wound, preferably comprises a flexible film which is no more than 20 to 250 micron thick.

10. The composition according to claim 1 wherein the carrier or base material of the strip comprises a soluble gel material, and is for example based upon on an organic gel, which could for example be a fish, animal, bovine or marine gelatine or vegetal gelatine-like product, a polysaccharide, a cellulosic material, pectin such as from fruits.

11. The composition according to claim 1 wherein additional active ingredient(s) are present selected from decongestants, lubricants, antibacterial and antiseptic compositions, anti-histamines, anti-inflammatory compositions, analgesics, open skin or wound healing or repair agents and other medicaments and non-medicaments.

12. The composition according to claim 1 which additionally includes adjuvants and the like such as vitamins for example selected from Ascorbic acid (vitamin C) which enhance the active ingredient effect.

13. The composition according to claim 1 wherein the active ingredient is present in an amount in the range 1-20 wt % and for example 2-10 wt %.

14. The composition according to claim 1 wherein the polysaccharide constituent is a negatively charged mixture of 70-90% D-Mannose 30-10% glucose, and 0-10% of other monosaccharides and is a plant derivative, for example Aloe Vera derivative, more preferably 2QR.

15. The composition according to claim 1 wherein the composition further includes means to stabilise the active ingredients in situ on the mucosa of the subject's throat or on open skin or a wound and to retain activity levels over a sustained period of time.

16. The composition according to claim 1 wherein a part or all of the active ingredients are encapsulated within encapsulation structures selected to provide the said degree of adhesion to the mucous membranes of the throat or open skin or wound, and adapted to release the active ingredients slowly over time in situ.

17. The composition according to claim 16 wherein the encapsulation structures comprise multilamellar microparticles.

18. A method of preparing a composition according to claim 1 for the controlled delivery of an active ingredient over time in situ comprising the steps of distributing at least one active ingredient as defined in within a soluble base, and providing as a strip of material for inter-oral or topical administration.

19. The method of claim 18 comprising combining the active ingredient and other components in solution with a solution of the base material and casting as a film to dry to a strip; alternatively a strip may be impregnated or coated with a dose of active ingredient and other components.

20. A method according to claim 18 wherein strips are made up in bulk films which are subsequently cut to size and shape.

21. A composition as defined in claim 1 for use in the alleviation of throat conditions or throat disorders, oral care conditions, snoring or sleep apnoea or wound or skin repair as hereinbefore defined.

22. (canceled)
